(19) United States
(12) Patent Application Publication
(10) Pub. No.: US 2025/0261899 A1
(43) Pub. Date: Aug. 21, 2025

(54) HOME-BASED LABOR ONSET DETECTION SYSTEM AND METHOD

(71) Applicant: GE Precision Healthcare LLC, Waukesha, WI (US)

(72) Inventors: Anu Antony, Bangalore (IN); Steven M. Falk, Baltimore, MD (US); Nagapriya Kavoori Sethumadhavan, Bangalore (IN); Rajendra Naik, Bangalore (IN)

(21) Appl. No.: 19/055,255

(22) Filed: Feb. 17, 2025

Related U.S. Application Data
(60) Provisional application No. 63/554,805, filed on Feb. 16, 2024.

(51) Int. Cl. A61B 5/00; A61B 5/1473; G16H 40/67

(52) U.S. Cl. CPC A61B 5/435; A61B 5/0013; A61B 5/1473; A61B 5/4362; A61B 5/6875; G16H 40/67; A61B 2503/02; A61B 2560/0406

(57) ABSTRACT

A system for detecting the onset of labor in a pregnant woman. The system includes a disposable cervical device configured to be self-inserted by the pregnant woman, the cervical device measures attributes of a cervix of the pregnant woman, and a smartphone application configured to receive data from the cervical device and to provide an indication of immediate onset of labor or risk of onset of labor within a predetermined time frame.

### CROSS-REFERENCE TO RELATED APPLICATIONS

The present application claims priority to U.S. Provisional Patent Application Ser. No. 63/554,805, filed on Feb. 16, 2024, and titled “HOME-BASED LABOR ONSET DETECTION SYSTEM AND METHOD,” which is incorporated herein by reference in its entirety.

### BACKGROUND

Embodiments disclosed in the present invention relate to a system and method for early detection of pre-term/term labor, and more specifically to a home-based labor onset detection system and method.

The timely detection of the onset of labor, particularly preterm labor, is a critical aspect of prenatal care. Preterm labor, defined as labor that begins before 37 weeks of gestation, can lead to premature birth, which is associated with a higher risk of neonatal morbidity and mortality. Additionally, the ability to distinguish between true labor and false labor is essential to prevent unnecessary hospital visits and to ensure that pregnant women receive appropriate care when true labor commences.

Traditionally, the detection and assessment of labor have required clinical evaluation by healthcare professionals. This often involves the use of various diagnostic tools and methods, such as the measurement of cervical changes through digital examination, the assessment of fetal heart rate, and the detection of specific biomarkers like Fetal Fibronectin (fFN) in cervical secretions. However, these methods necessitate a visit to a healthcare facility, which may not be readily accessible to all women, especially those in remote or underserved areas.

Moreover, the current clinical methods for labor detection involve subjective assessments, such as the Bishop Score, which can suffer from high intra- and inter-observer variability. Additionally, these methods can be uncomfortable for the patient and may not provide a continuous or real-time assessment of labor progression.

The advent of home-based medical devices has begun to address some of these challenges by allowing for self-monitoring of various health conditions. However, there remains a significant gap in the availability of a comprehensive, user-friendly, and non-invasive home-based system that can accurately detect the onset of labor, assess the risk of preterm labor, and provide real-time data to healthcare providers for timely intervention.

Therefore, a novel solution that can overcome the limitations of existing technologies and further help in early detection of pre-term/term labor would be highly beneficial to both patients and healthcare providers.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

In one embodiment, a system for detecting the onset of labor in a pregnant woman, including a disposable cervical device configured to be self-inserted by the pregnant woman, the cervical device measures attributes of a cervix of the pregnant woman, and a smartphone application configured to receive data from the cervical device and to provide an indication of immediate onset of labor or risk of onset of labor within a predetermined time frame.

In another embodiment, a method for detecting the onset of labor in a pregnant woman, the method including instructing the pregnant woman to self-insert a cervical device, measuring aspects of a cervix of the pregnant woman with the cervical device, transmitting the measurements to a smartphone application, and analyzing the transmitted data with the smartphone application to provide an indication of labor onset or risk thereof.

Various other features, objects, and advantages of the invention will be made apparent from the following description taken together with the drawings.

### BRIEF DESCRIPTION OF THE DRAWINGS

The present disclosure is described with reference to the following Figures.

FIG. 1 schematically depicts an exemplary home-based testing kit for use with a mobile application according to an embodiment of the invention.

FIG. 2 provides a flow chart for using the home-based testing kit of FIG. 1.

FIG. 3 provides a flow chart for using the home-based testing kit of FIG. 1.

FIG. 4 provides a flow chart for using the home-based testing kit of FIG. 1.

### DETAILED DESCRIPTION

One or more specific embodiments will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present embodiments, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments. Furthermore, the terms “circuit” and “circuitry” and “controller” may include either a single component or a plurality of components, which are either active and/or passive and are connected or otherwise coupled together to provide the described function.

It is estimated that more than 1 in 10 babies are born prematurely worldwide. Moreover, premature babies can face lifelong health-related disabilities, such as difficulties in learning or hearing and vision loss in addition to neurodevelopmental impairments leading to a compromised quality of life. By monitoring of uterine contractions, cervical effacement, cervical dilation, fetal heart rate and presence of Fetal Fibronectin, physicians can determine if the pregnant woman is in labor or will soon be in labor and can determine the stage of labor. A pregnant woman at home is unable to assess cervical effacement and dilation to determine if and when to go to the hospital.

Studies have shown that long distances from hospitals, such as in rural areas, are associated with higher mortality and a lack of healthcare access. Furthermore, since it is difficult to prevent premature births, mitigating the effects and consequences of premature births on the fetus and the mother is the best solution found so far. To prevent premature births and mitigate and reduce their consequences, we need a better understanding of this issue. One of the key factors in mitigating premature birth issues is the early detection of labor. Early detection can help provide medical intervention and achieve the best childbirth outcome and treatment. Furthermore, early detection helps mitigate the health risks for the foetus and the mother and reduces the treatment cost of premature birth complications.

Today, a pregnant woman in a remote area or a maternity desert cannot go to a clinic for a quick check if she doubts that she may be going into premature labor. Also, in pregnancies where premature labor is anticipated, it is difficult to know when the mom may go into premature labor. This could result in a home birth without medical facilities or in unnecessary visits to the hospital. This invention solves the problem by providing the pregnant woman with a disposable self-test device that can provide an indication of onset/risk of onset of labor.

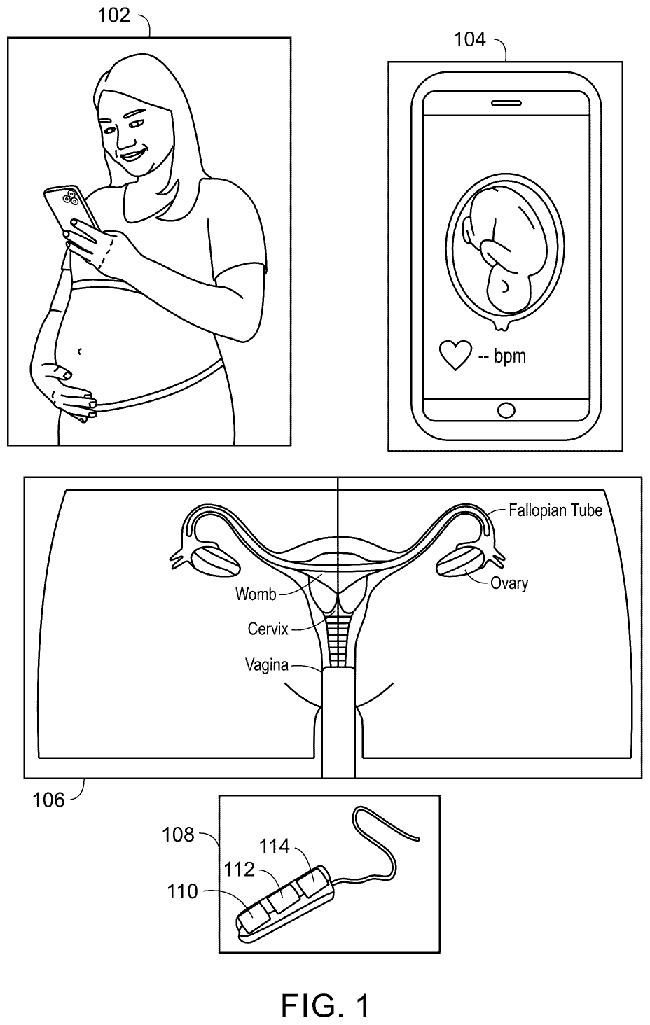

FIG. 1 shows a depiction of the present technique. Specifically, FIG. 1 shows a pregnant woman 102 using a mobile application 104 to differentiate between true labor and false labor. Additionally, the mobile application 104 assists the pregnant woman 102 in determining when to go to a hospital. In general, the mobile application 104 helps in early and reliable detection of onset/risk of onset of labor for the pregnant woman 102. FIG. 1 also shows a home-based screening kit 108 which may have a form factor of a disposable tampon. In other embodiments, the home-based screening kit 108 may define an alternative cervical device. The screening kit 108 may be inserted in a cervix 106 of the pregnant woman 102. The screening kit 108 may include a miniature inbuilt camera 110 to visualize the dilatation of the cervix, a thin Ultrasound patch 112 to measure the cervical length and detect the Fetal Heart rate, and a sensor 114 to detect the presence of Fetal Fibronectin (fFN) in cervical secretions. The camera 110, the patch 112, and the sensor 114 may be disposed at any location on the screening kit 108. In other embodiments, the screening kit 108 may include additional and/or alternative measurement devices in order to measure aspects of the cervix.

The screening kit 108 can be used by the below category of pregnant mothers without any assistance: 1) High risk pregnant mother (with previous history of miscarriages and preterm births) who might be at a risk for preterm labor, 2) Pregnant mother who is likely to be in false labor and 3) Term mother for an initial assessment of timing of labor and delivery.

The camera images and other sensor parameters obtained by the screening kit 108 are shared with a remote physician via the app 104 on the smartphone. Additionally based on the parameters, an indication for (i) immediate onset of labor (ii) risk of onset of labor in the next 7-14 days is provided. Based on this, the pregnant woman 102, if in labor (as indicated by the device), can go to the hospital. If the device indicates that there is a risk of the pregnant woman 102 going into labor in the next few days, the pregnant woman 102 can retest the next day or the day after and go to the hospital as needed in discretion with the advice from the remote physician.

The screening kit 108 is disposable. Therefore, the user does not have to worry about sterilization or disinfection before reuse. Further, the proposed solution uses FHR to determine fetal distress.

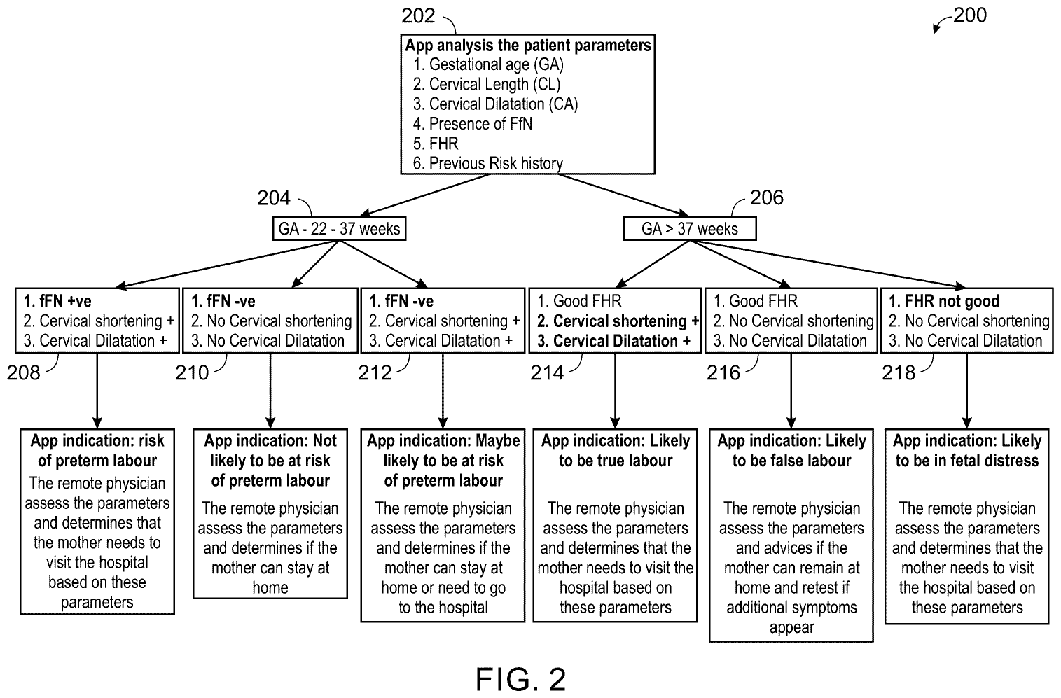

FIG. 2 shows a flowchart 200 depicting an algorithm in the mobile app 104 of FIG. 1. The mobile app 104 may receive some of the data from patient history and other data from the cervical device 108. The mobile app 104 is designed to analyze a plurality of patient parameters in step 202. The plurality of patient parameters include Gestational age (GA), Cervical length (CL), Cervical Dilatation (CA), Presence of Fetal Fibronectin (fFN), Fetal Heart Rate (FHR), Previous Risk History. In other embodiments, the plurality of patient parameters may include additional and/or alternative parameters.

Based on the plurality of parameters, the algorithm 200 provides different indications. For example, in step 202, if Gestational age is of 22 to 37 weeks, then the algorithm moves to one of the steps 208, 210 and 212. The step 208 corresponds to a case when fFN is positive and cervical shortening and cervical dilatation are present. Based on step 208 the mobile app 104 indicates a risk of preterm labor. The remote physician assesses the parameters and may determine that the mother needs to visit the hospital.

Further, step 210 corresponds to a case when fFN is negative, and there is no cervical shortening or dilatation. In this case, the mobile app 104 indicates that the mother is not likely to be at risk of preterm labor. The remote physician assesses the parameters and may determine that the mother can stay at home. Moreover, step 212 corresponds to a case where fFN is negative, but cervical shortening and cervical dilatation are present. In this scenario, the mobile app 104 indicates that the mother may be likely to be at risk of preterm labor. The remote physician assesses the parameters and determines if the mother can stay at home or needs to go to the hospital.

On the other hand, if in step 202 it is determined that the Gestational age is greater than 37 weeks, then the algorithm moves to one of the steps 214, 216 and 218. The step 214 corresponds to a case where there is a good FHR, cervical shortening, and cervical dilatation. In this case, the mobile app 104 indicates that the mother is likely to be in true labor. The remote physician assesses the parameters and determines that the mother needs to visit the hospital.

Further, step 216 corresponds to a case where there is good FHR, but no cervical shortening or dilatation. For this case, the mobile app 104 indicates that the mother is likely to be in false labor. The remote physician assesses the parameters and advises if the mother can remain at home and retest if additional symptoms appear. Moreover, step 218 corresponds to a case when the FHR is not ideal, and there is no cervical shortening or dilatation. Based on this, the mobile app 104 indicates that the fetus is likely to be in distress. The remote physician assesses the parameters and determines that the mother needs to visit the hospital.

FIG. 2 emphasizes the role of the mobile app 104 in providing a preliminary assessment that can guide the pregnant woman's decision to seek medical attention, with the remote physician's input being a crucial part of the decision-making process.

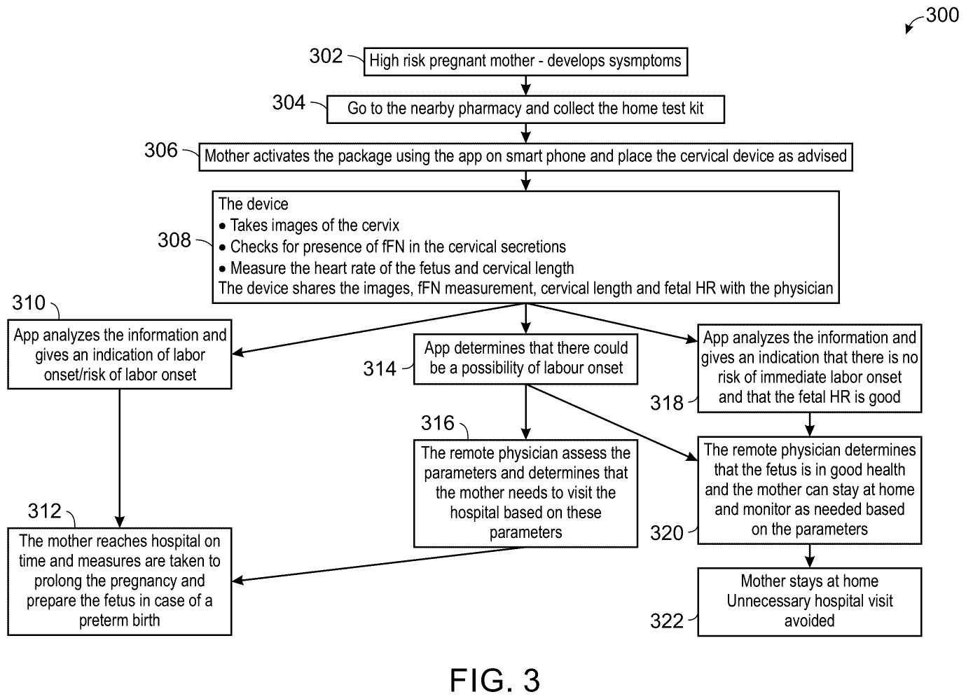

FIG. 3 shows a flowchart 300 for a process involving a high-risk pregnant mother who may develop symptoms of pre-term labor. The flow chart 300 emphasizes the dual functionality of the home test kit 108: it not only provides an assessment of the risk of labor onset but also communicates vital information to a physician who can make a well-informed decision about the need for hospital intervention. This scenario demonstrates how the test kit 108 can be used by a high-risk pregnant mother to make timely and informed decisions about seeking medical care.

In this process, at step 302, a high-risk mother develops symptoms and so at step 304, the mother goes to a nearby pharmacy and collects the home test screening kit 108. At step 306, the mother activates the package using the mobile app 104 on her smartphone and places the cervical device as advised. At step 308, the screening kit 108 performs several functions. For example, the screening kit 108 takes images of the cervix via the camera 110, checks for the presence of fFN in the cervical secretions via the sensor 114, and measures the fetal heart rate and cervical length via the patch 112. The screening kit 108 then shares the images, fFN measurement, cervical length, and fetal heart rate with the physician. At steps 310, 314 and 318, the mobile app 104 analyzes the information and provides an indication of labor onset or risk of labor onset (step 310), indication of possibility of labor onset (step 314) or indication that there is no risk of immediate labor onset and that the fetal HR is good (step 318).

Based on indication of labor onset/risk of labor onset in step 310, the mother reaches a hospital on time, and measures are taken to prolong the pregnancy and prepare the fetus in case of a preterm birth at step 312. On the other hand, if there was indication that there is no risk of immediate labor onset at step 318 then at step 320, the remote physician may determine that the fetus is in good health and the mother can stay at home and monitor as needed based on the parameters. Accordingly, at step 322, the mother stays at home and avoids an unnecessary hospital visit.

Further, if there was only an indication of possibility of labor onset as in step 314, then at step 316, the remote physician assesses the parameters and then decides whether the mother should follow step 312 or steps 320-322.

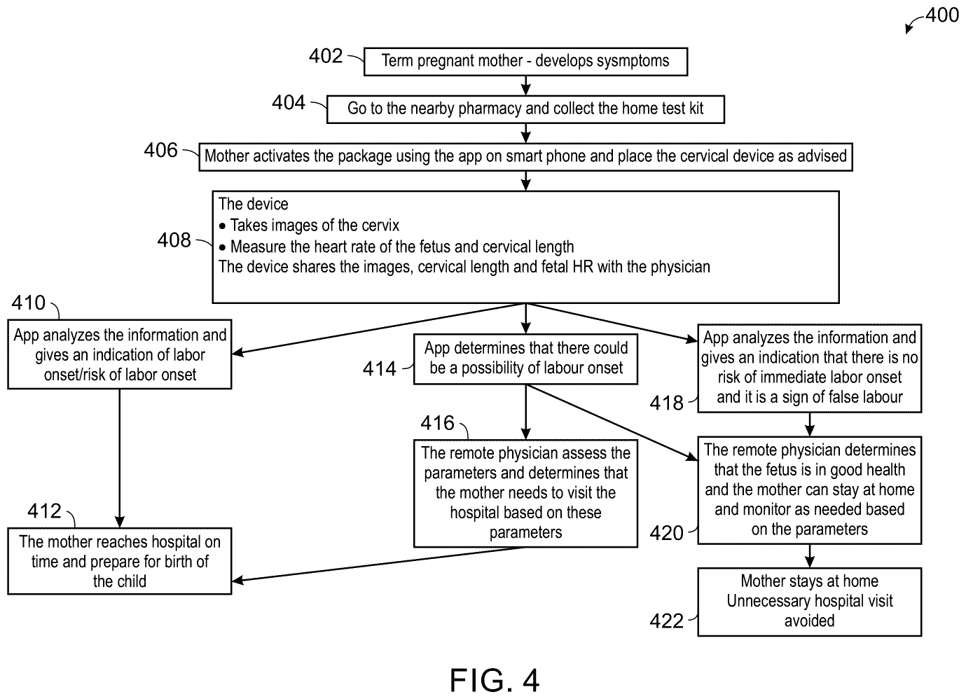

FIG. 4 shows a flowchart 400 for a process involving a term pregnant mother who may develop symptoms. The flow chart 400 emphasizes how the home test kit 108 can assist a term pregnant mother in determining whether she is experiencing true labor, thereby facilitating timely and appropriate medical intervention. It also illustrates the kit's role in preventing unnecessary hospital visits when the symptoms are indicative of false labor, contributing to a more efficient and patient-centered approach to labor management.

In this process, at step 402, a term pregnant mother develops symptoms and so at step 404, the mother goes to a nearby pharmacy and collects the home test screening kit 108. At step 406, the mother activates the package using the mobile app 104 on her smartphone and places the cervical device as advised. At step 408, the screening kit 108 performs several functions. For example, the screening kit 108 takes images of the cervix via the camera 110, measures the heart rate of the fetus via the patch and cervical length via the patch 112. The screening kit 108 then shares the images, cervical length, and fetal heart rate with the physician. At steps 410, 414 and 418, the mobile app 104 analyzes the information and provides an indication of labor onset or risk of labor onset (step 410), indication of possibility of labor onset (step 414) or indication that there is no risk of immediate labor onset and that it is a sign of false labor (step 418).

Based on indication of labor onset/risk of labor onset in step 410, the mother then reaches the hospital on time to prepare for the birth of the child. On the other hand, if there was indication that there is no risk of immediate labor onset and it is a sign of false labor at step 418 then at step 420, the remote physician may determine that the fetus is in good health and the mother can stay at home and monitor as needed based on the parameters. Accordingly, at step 422, the mother stays at home and avoids an unnecessary hospital visit.

Further, if there was only an indication of possibility of labor onset as in step 414, then at step 416, the remote physician assesses the parameters and then decides whether the mother should follow step 412 or steps 420-422.

The benefits of the present technique are it helps in determining the need for quicker access to the hospital for better prevention and management care. The technique also reduces the risk of birth of premature babies and helps to prolong pregnancy to term. The birth of a child preterm has also been seen to have economic implications due to the costs of the hospitalisation of pregnant patients with forecasted preterm birth. In addition to this, there also emerge cost implications for false positives in the women who ended up not giving birth during their stay in the hospital. Furthermore, post care implications arise for the false negatives in women who conversely were not given early admittance to hospital but eventually ended up as a preterm patient.

Further, the present technique helps the mother in detecting true labor. The technique also helps to reduce unwanted visits to hospitals in case of a false labor. Moreover, early and reliable detection of cervical dilatation at home helps the mother to plan and prepare for delivery ahead of time and avoid emergencies which leads to unwanted complications.

The present invention seeks to fill gaps in existing technologies by introducing a novel home-based screening kit 108 that enables pregnant women to self-test for the onset of labor. This screening kit 108 integrates a disposable cervical device with advanced sensing technologies, including a miniature camera, an ultrasound patch, and a sensor for Fetal Fibronectin (fFN) detection. The data collected by the device is analyzed by a smartphone application, which provides an immediate assessment of labor status and communicates with a remote physician for further evaluation. This innovative approach aims to improve prenatal care by empowering expectant mothers with the tools to monitor their labor progression in the comfort of their homes, thereby enhancing the safety and well-being of both mother and child.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A system for detecting the onset of labor in a pregnant woman, comprising:
a disposable cervical device configured to be self-inserted by the pregnant woman, the cervical device measures attributes of a cervix of the pregnant woman; and
a smartphone application configured to receive data from the cervical device and to provide an indication of immediate onset of labor or risk of onset of labor within a predetermined time frame.

2. The system of claim 1, wherein the cervical device includes a miniature camera for visualizing cervical dilation.

3. The system of claim 1, wherein the cervical device includes an ultrasound patch for measuring cervical length and detecting fetal heart rate.

4. The system of claim 1, wherein the cervical device includes a sensor for detecting the presence of Fetal Fibronectin (fFN) in cervical secretion.

5. The system of claim 1, wherein the cervical device has a form factor of a disposable tampon.

6. The system of claim 1, wherein the smartphone application is further configured to analyze patient parameters including gestational age, cervical length, cervical dilatation, presence of fFN, fetal heart rate, and previous risk history to assess the risk of preterm labor.

7. The system of claim 1, wherein the smartphone application is further configured to communicate with a remote physician to provide an assessment based on the received data.

8. The system of claim 1, wherein the indication of labor onset or risk thereof is based on a combination of detected presence of fFN, cervical shortening, and cervical dilatation.

9. The system of claim 1, wherein the disposable cervical device is configured for use by a high-risk pregnant mother with a previous history of miscarriages or preterm births or a pregnant mother likely to be in false labor.

10. The system of claim 1, wherein the disposable cervical device is configured for use by a term mother for an initial assessment of timing of labor and delivery.

11. A method for detecting the onset of labor in a pregnant woman, the method comprising:
instructing the pregnant woman to self-insert a cervical device;
measuring aspects of a cervix of the pregnant woman with the cervical device;
transmitting the measurements to a smartphone application; and
analyzing the transmitted data with the smartphone application to provide an indication of labor onset or risk thereof.

12. The method of claim 11, wherein measuring aspects of the cervix includes capturing images of the cervix, measuring cervical length, detecting fetal heart rate, and detecting the presence of fFN in cervical secretions using the cervical device.

13. The method of claim 11, further comprising communicating the analyzed data to a remote physician for assessment and advice.

14. The method of claim 11, wherein the indication of labor onset or risk thereof includes an assessment of fetal distress based on fetal heart rate and cervical conditions.

15. The method of claim 11, wherein the cervical device has a form factor of a disposable tampon.

16. The method of claim 11, further comprising analyzing patient parameters including gestational age, cervical length, cervical dilatation, presence of fFN, fetal heart rate, and previous risk history to assess the risk of preterm labor with the smartphone application.

17. The method of claim 11, wherein the indication of labor onset or risk thereof is based on a combination of detected presence of fFN, cervical shortening, and cervical dilatation.

18. The method of claim 11, wherein the disposable cervical device is configured for use by a high-risk pregnant mother with a previous history of miscarriages or preterm births or a pregnant mother likely to be in false labor.

19. The method of claim 11, wherein the disposable cervical device is configured for use by a term mother for an initial assessment of timing of labor and delivery.
